(19) United States
(12) Patent Application Publication
(10) Pub. No.: US 2022/0202730 A1
(43) Pub. Date: Jun. 30, 2022

(54) 5-AMINOLEVULINIC ACID FOR THE LOCAL TREATMENT OF INFLAMMATORY BOWEL DISEASE

(71) Applicant: Intract Pharma Limited, London (GB)

(72) Inventors: Abdul Waseh Basit, London (GB); Vipul Yadav, London (GB); Satofumi Kawata, Tokyo (JP); Motoyasu Tomioka, Tokyo (JP); Yasufumi Wada, Tokyo (JP)

(21) Appl. No.: 17/607,739

(22) PCT Filed: Apr. 29, 2020
(86) PCT No.: PCT/EP2020/061949

§ 371 Date: Oct. 29, 2021

(30) Foreign Application Priority Data
Apr. 29, 2019 (GB) 1905940.1

(51) Int. Cl. A61K 9/50; A61K 31/197; A61K 9/00; A61K 9/28

(52) U.S. Cl. CPC A61K 9/5026; A61K 31/197; A61K 9/5036; A61K 9/286; A61K 9/2846; A61K 9/0031

(57) ABSTRACT

The present invention relates to the therapeutic use of compositions containing 5-aminolevulinic acid for the local treatment of inflammatory bowel disease, including but not restricted to ulcerative colitis and Crohn's disease.

### FIELD OF THE INVENTION

The present invention relates to the therapeutic use of compositions containing 5-aminolevulinic acid for the local treatment of inflammatory bowel disease, including but not restricted to ulcerative colitis and Crohn's disease.

### BACKGROUND

Inflammatory bowel disease (IBD) is a chronic relapsing inflammatory disease of the gastrointestinal (GI) tract that is characterized by long term impairment of the GI structure and function with symptoms such as abdominal pain, intestinal bleeding and diarrhoea. IBD has two main forms, ulcerative colitis (UC) and Crohn's disease (CD). In UC, inflammation is confined mainly to the mucosal region of the distal colon and rectum, while inflammation in CD can occur throughout the GI tract in patches and is transmural in nature affecting all layers of the intestinal tissue.

Current first line therapy includes aminosalicylates such as mesalazine or 5-aminosalicylic acid (5-ASA) which are available in various formulations that release the drug into the gut lumen through pH, time or carrier dependent trigger mechanisms. However, the high dose of mesalazine (3-4 g/day) can be problematic for patients due to high frequency of dosing leading to poor compliance and adherence to therapy leaving the patients at risk of going into relapse. 5-ASA is less effective in maintaining remission in CD and ineffective in steroid induced remission. Drugs, including sulfasalazine, olsalazine and balsalazide, which are metabolised to 5-ASA through a bacteria cleavable bond, and corticosteroids such as prednisolone and budesonide, are also prescribed in patients with IBD. However, these are associated with risk of adverse effects such as diarrhoea, nausea, epigastric pain, headache and rash.

The therapy for IBD has rapidly evolved with the introduction of novel biologics-based treatments that target specific inflammatory cytokines in the body to reduce inflammation and maintain remission. However, significant adverse effects related to the high systemic exposure of biologics and frequent need for injections including hospitalization for intravenous infusions demands innovations for better therapy for patients with IBD.

There is a need for an alternative first line therapy for the treatment of CD and UC with safe and efficacious small molecules at doses and frequency that are patient friendly and improve adherence to therapy.

### SUMMARY OF THE INVENTION

The present invention relates to the surprising finding that in mice with dextran sodium sulfate (DSS) induced colitis treatment with the delivery of 5-aminolevulinic acid (5-ALA) directly to the inflamed colon can reduce the production of key inflammatory biomarkers significantly more effectively than is observed following delivery of the compound orally.

5-aminolevulinic acid (5-ALA), a naturally occurring amino acid and a precursor of heme and protoporphyrin IX (PpIX), has been implicated in inflammatory and autoimmune diseases when administered in combination with sodium ferrous citrate (SFC), thought to be brought about by anti-inflammatory properties elicited by expression of heme oxygenase-1 (HO-1), the enzyme which catalyses a rate limiting step in the oxidative degradation of heme into free iron, biliverdin and bilirubin (Fujino et al. 2016).

In the present studies examining colonic delivery of 5-ALA via the oral route no significant difference in the expression of HO-1, biliverdin and bilirubin is observed in the colon tissue and plasma of the animals treated with oral and intra-colonic 5-ALA. Significantly enhanced expression of PpIX was observed in the plasma after oral dosing, but not intra-colonic dosing, suggesting metabolism of 5-ALA occurred effectively only after oral dosing due to absorption of 5-ALA into the systemic circulation from the small intestine. Hence, the present invention reports a surprising observation that topical colonic delivery of 5-ALA leads to superior anti-inflammatory effects compared to oral dosing even though there is no enhanced metabolism of 5-ALA into the proposed anti-inflammatory metabolites in the colon tissue or plasma after colon delivery.

Accordingly, the invention provides a pharmaceutical composition comprising 5-aminolevulinic acid (5-ALA) or a pharmaceutically acceptable salt thereof, adapted for topical administration to the lower gastrointestinal tract of a human or animal.

The pharmaceutical composition may be adapted for rectal administration.

Alternatively the pharmaceutical composition may be adapted for delayed release oral administration.

The pharmaceutical composition may further comprise an enteric coating.

The pharmaceutical composition may comprise a solid oral dosage form with a core and a coating for the core, the core comprising 5-ALA or a pharmaceutically acceptable salt thereof, and the coating comprising a mixture of a digestible polysaccharide and a film-forming material which has a pH threshold at pH 6.0 or above. The digestible polysaccharide may be selected from the group consisting of starch; amylose; amylopectin; chitosan; chondroitin sulfate; cyclodextrin; dextran; pullulan; carrageenan; scleroglucan; chitin; curdulan and levan. Preferably the polysaccharide is starch, amylose or amylopectin.

Preferably the film-forming is an acrylate polymer, a cellulose polymer or a polyvinyl-based polymer. Preferably the film-forming is selected from cellulose acetate phthalate; cellulose acetate trimellitate; hydropropylmethylcellulose acetate succinate; and polyvinyl acetate phthalate. More preferably the film-forming is a co-polymer of a (meth)acrylic acid and a (meth)acrylic acid C₁₋₄ alkyl ester.

A pharmaceutical composition as claimed in any preceding claim, for use in the prophylaxis or treatment of inflammatory bowel disease, irritable bowel disease, autoimmune disease, constipation, diarrhoea, infection, or cancer. Preferably the inflammatory bowel disease is Ulcerative colitis or Crohn's disease.

The present invention also provides a method of treating inflammatory bowel disease, irritable bowel disease, autoimmune disease, constipation, diarrhoea, infection, or cancer comprising administering 5-aminolevulinic acid (5-ALA) or a pharmaceutically acceptable salt thereof to the lower gastrointestinal tract of a human or animal. Preferably the inflammatory bowel disease is Ulcerative colitis or Crohn's disease. Preferably the method comprises administering a pharmaceutical composition of the invention. The composition is administered topically to the mucous membrane of the lower gastrointestinal tract.

Surprisingly, the present inventors have found using in vitro analysis employing human colonic tissues and fluids, that 5-ALA is very stable in the colonic environment and is readily able to penetrate effectively into colon tissue. As is observed by in vivo analysis in DSS-treated mice, no metabolism of 5-ALA to PpIX, biliverdin and bilirubin in is observed in human colonic fluid or colonic tissue specimens in vitro.

### DETAILED DESCRIPTION OF THE INVENTION

5-Aminolevulinic Acid

As used herein, “5-ALA” refers to the naturally occurring amino acid 5-aminolevulinic acid or a pharmaceutically acceptable salt thereof. The compound, described below, has previously been utilized as a precursor of a photosensitizer PpIX for performing photodynamic diagnosis and therapy to confirm and kill tumor cells. 5-ALA is converted into PpIX and heme in the mitochondria with the insertion of iron catalysed by ferrochelatase. HO-1 further catalyses the conversion of heme into carbon monoxide (CO), biliverdin and bilirubin. 5-ALA (CAS No. 106-60-5) has the general formula as below:

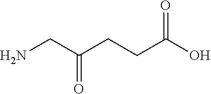

A “pharmaceutically acceptable salt” is one which is safe for use in mammals and retain the desired biological activity. Suitable salts include but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Preferably, the salt is hydrochloride.

“5-ALA” as used herein also encompasses precursors or prodrugs of 5-aminolevulinic acid. A “precursor/prodrug” is a compound which is metabolised to 5-aminolevulinic acid following administration in the intestinal tract, e.g. through the action of bacteria found in the gastrointestinal tract, in particular the lower gastrointestinal tract.

“5-ALA” as used herein also encompasses derivatives of 5-aminolevulinic acid and their pharmaceutically acceptable salts. Derivatives include esters, where the —OH group of 5-aminolevulinic acid is replaced with —O—R wherein R is an alkyl group with up to 8 carbons atoms. Preferably the alkyl group has 1, 2, 3, 4, 5, 6, 7 or 8 carbon atoms. The alkyl group is preferably unbranched. Suitable esters include methyl aminolevulinic acid and ethyl aminolevulinic acid, and preferred salts include methyl aminolevulinate hydrochloride and ethyl aminolevulinate hydrochloride.

Pharmaceutical Formulations

The pharmaceutical compositions of the invention provide delivery of the 5-ALA to the lower gastrointestinal tract. The composition is administered topically to the mucous membrane of the lower gastrointestinal tract.

As used herein “lower gastrointestinal tract” refers to gastrointestinal tract after the stomach. This includes the small intestine and large intestine. The small intestine is made up of the duodenum and ileum, while the large intestine is also known as the colon. Preferably, the pharmaceutical compositions deliver the 5-ALA to the ileum and/or colon.

The 5-ALA is delivered topically to the surface mucosa of the lower gastrointestinal tract. This can be achieved through rectal administration e.g. as a suppository or enema; or through a delayed released oral formulation, wherein the 5-ALA is released from the formulation in the lower gastrointestinal tract.

The pharmaceutical composition may be prepared as a liquid but is preferably in a solid or semi-solid form, and most preferably in a form which is suitable for oral or rectal administration. The composition may also be in the form of a lotion, cream, foam, emulsion or gel. Such formulations may be prepared by a number of known methods established in the art.

Pharmaceutical compositions in the present invention that are suitable for oral administration may be presented either in the form of tablets, capsules, mini-tablets, pellets, powders, granules, microparticles, nanoparticles or hydrogels.

Compositions of the present invention suitable for oral administration may be presented as discrete units such as capsules, tablets, mini-tablets, or pellets, or as powders, granules or crystals. In a solid composition, the minimum diameter of each particle is typically at least 10⁻⁴ m, usually at least 5×10⁻⁴ m and, preferably at least 10⁻³ m. The maximum diameter is usually no more than 30 mm, typically no more than 20 mm and, preferably, no more than 10 mm. In preferred embodiments, the particle has a diameter from about 0.2 mm to about 15 mm, preferably from about 1 mm to about 4 mm (e.g. for pellets or mini-tablets) or from about 6 mm to about 12 mm (e.g. for certain tablets or capsules). The term “diameter” refers to the largest linear dimension through the particle.

The compositions of the invention may comprise one or more further conventional excipients as required, such as binders, extenders, disintegrants, diluents and lubricants. Excipients used in solid forms include for example, microcrystalline cellulose, dicalcium phosphate, starch, magnesium stearate, calcium sulfate, sorbitol, glucose and/or lactose and/or other excipients known in the art. Suitable binders include starch, gelatine, natural sugars such as glucose or beta-lactose, corn sweeteners, natural and synthetic gums such as acacia, tragacanth or sodium alginate, carboxymethylcellulose, polyethylene glycol, waxes and the like. Disintegrators include without limitation starch, methylcellulose, agar, bentonite, xanthan gum and the like. Fast dissolving diluents include mannitol, lactose, sucrose and/or cyclodextrins. Lubricants, glidants, flavours, colouring agents and stabilizers may also be added for ease of fabrication and use. Lubricants include sodium oleate, sodium stearate, magnesium stearate, sodium benzoate, sodium acetate, sodium chloride.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of 5-ALA. Preferred examples of coatings are given below.

Capsules may have solid, semi-solid or non-solid contents. Exemplary contents for capsules may include suspensions which may contain one or more suitable excipients. For example, the composition may comprise microcrystalline cellulose for imparting bulk, alginic acid or sodium alginate as a suspending agent, and/or methylcellulose as a viscosity enhancer, as well as any of the solid or semi-solid forms above.

Formulations for rectal administration may be presented as a suppository with a suitable carrier such as cocoa butter, synthetic glyceride esters or polyethylene glycol. Such carriers are typically solid at ordinary room temperatures (up to 25° C.) but liquefy and/or dissolve in the rectal cavity to release the drug.

The pharmaceutical composition may take the form of an enema formulation such as a liquid or foam enema which is rectally administered to the lower colon. The enema formulations typically comprise the 5-ALA dissolved or dispersed in a suitable flowable carrier vehicle, such as deionised and/or distilled water. The formulation can be thickened with one or more thickeners. They may also contain a buffer, and can also comprise an effective amount of a lubricant such as a natural or synthetic fat or oil, e.g. a tris-fatty acid glycerate or lecithin. Non-toxic non-ionic surfactants can also be included as wetting agents and dispersants. A buffer is preferably added to the liquid or foam enema of 5-ALA to stabilise the pH. The pH is preferably 3.5 to 7.5, especially 6.5 to 7.5.

Unit doses of enema formulations can be administered from pre-filled bags or syringes. In the case of a pressurised enema formulation the carrier vehicle may also comprise an effective amount of a foaming agent such as n-butane, propane or i-butane, or the foaming agent/propellant could be held separately from the composition such as in a bag-in-bag or bag-in-can system as described in WO-A-9603115 (incorporated herein by reference). Enema foams may also comprise expanding agents and foam-stabilisers.

The volume of a liquid enema is typically 50-200 cm³, preferably about 100 cm³. The volume of a foam enema is typically 20 to 40 cm³. A suitable dosage of 5-ALA in the enema as administered is 4 mg/ml to 20 mg/ml, preferably 6 mg/ml to 10 mg/ml.

Preferred unit dosage formulations are those containing an effective dose, or an appropriate fraction thereof, of the active ingredient. Release from certain formulations may be sustained, if the composition contains suitable controlled-release excipients. However, in preferred formulations, release is pulsatile.

The compositions according to the invention will typically comprise a therapeutically effective amount of 5-ALA which may be from 0.01 wt % to 99 wt %, based on the total weight of the composition. The actual dosage would be determined by the skilled person using common general knowledge. However, by way of example, “low” dose formulations typically comprise no more than 20 wt % 5-ALA, and preferably comprise from 1 wt % to 20 wt %, more preferably 2 wt % to 10 wt % 5-ALA, e.g. 5 wt % 5-ALA. “High” dose formulations typically comprise at least 40 wt % 5-ALA, and preferably from 45 wt % to about 85 wt %, e.g. 50 wt % or 80 wt %.

Whilst 5-ALA may be used as the sole active ingredient in a composition according to the invention, it is also possible for 5-ALA to be used in combination with one or more further therapeutic agents. Thus, the invention also provides a composition according to the invention containing one or more therapeutic agents in addition to 5-ALA. If desired, the composition according to the invention may be administered together with a further composition, by simultaneous, sequential or separate administration. Preferably, the pharmaceutical composition further comprises sodium ferrous citrate (SFC).

Except where the context requires otherwise, throughout this specification and claims, any reference to a pharmaceutical composition in solid or semi-solid form should be understood to include individual solid or semi-solid particles or unit forms which are solid or semi-solid throughout, as well as those having a solid or semi-solid exterior and a non-solid, for example liquid or gel, interior. For example, a capsule may have liquid or gel contents.

Enteric Coatings

In a particularly preferred embodiment of the invention, the composition is adapted for selective release of the 5-ALA in the lower gastrointestinal tract, especially the ileum and/or the colon, suitably following rectal or, especially, oral administration. This may be accomplished by the use of particular coatings. The compositions of the invention may be delayed release oral (DRO) compositions. The DRO compositions pass through the stomach substantially unaltered and deliver the active ingredient to the lower gastrointestinal tract, typically the ileum and/or colon (i.e. the site of the diseased mucosa).

The compositions according to the invention may have an enteric coating. Enteric coatings protect the active ingredients in a composition from attack and degradation in the stomach, but dissolve and release the contents of the dosage form within the intestines, usually due to the change in pH. Suitable enteric coatings are well known in the art. The optimal coating for any particular formulation depends on the exact intended use, and coatings may be tailored to release the active ingredient in a particular region of the intestines, or at a particular time following ingestion. Preferably, the composition of the present invention is in a solid or semi-solid form which comprises an enteric coating. Useful enteric coatings are those which remain intact in the low pH environment of the stomach, but readily dissolve when the optimum pH for dissolution is reached. This can vary between pH 3 to 7.5, preferably 5 to 7, depending on the chemical composition of the coating. The thickness of the coating required will depend on the solubility of the coating and the intended site to be treated. Typically the coating is 25 to 200 μm, especially 75 to 150 μm.

The composition of the invention is adapted for release of the 5-ALA to the part of the lower gastrointestinal tract where the disease is prevalent. Typically the enteric coating should dissolve in the pH of the jejunum (about pH 5.5), ileum (about pH 6) and/or colon (pH 6-7) to that the majority of the 5-ALA is released at the desired site.

Such a formulation may if desired contain one or more intermediate layers between the active ingredient and the outer enteric coating. In this case, it is possible for a composition of the invention to release a portion of its contents at one particular region of the intestines, and a further portion of its contents at a lower region of the intestines. The composition of the invention delivers at least a portion of the 5-ALA to the lower gastrointestinal tract. Preferably the composition delivers the majority of the 5-ALA to the lower gastrointestinal tract, for example 80% or more, 85% or more, 90% or more, preferably at least 95% of the total 5-ALA in the composition. Preferably the 5-ALA is delivered to the ileum and/or colon.

Delivery to the Ileum and/or Colon

Of particular interest in the context of the present invention are formulations which provide release only within a specific part of the GI tract, particularly the ileum and/or, the colon. WO 2007/122374 (the contents of which are incorporated herein by reference) describes such formulations, and these form preferred compositions of the invention. Accordingly, the invention further provides a composition comprising a solid oral dosage form with a core and a coating for the core, the core comprising 5-ALA; the coating comprising a mixture of a digestible polysaccharide and a film-forming polymeric material which has a solubility threshold at pH 5 or above.

The digestible polysaccharide is susceptible to attack by intestinal bacteria. Preferably the digestible polysaccharide is selected from the group consisting of starch; amylose; amylopectin; chitosan; chondroitin sulfate; cyclodextrin; dextran; pullulan; carrageenan; scleroglucan; chitin; curdulan and levan. Preferably the digestible polysaccharide is starch, amylose or amylopectin.

Starches are usually extracted from natural sources such as cereals; pulses; and tubers. Suitable starches for use in the present invention are typically food grade starches and include rice starch; wheat starch; corn (or maize) starch; pea starch; potato starch; sweet potato starch; tapioca starch; sorghum starch; sago starch; and arrow root starch.

The film-forming polymeric material is an enteric material which has a pH threshold which is the pH below which it is insoluble and at or above which it is soluble. The pH of the surrounding medium triggers dissolution of the second material. The normal pH of gastric juice is usually in the range of 1 to 3, while the pH of intestinal juice gradually increases from about 5.5 in the duodenum to about 7 to 8 in the colon. The second material preferably has a pH threshold of 6.0 or greater, especially 7 or greater. The pH threshold at which a material becomes soluble may be determined by a simple titration technique which would be part of the common general knowledge to the person skilled in the art.

The film-forming polymeric material may be an acrylate polymer, a cellulose polymer or a polyvinyl-based polymer. Examples of suitable cellulose polymers include cellulose acetate phthalate (“CAP”); cellulose acetate trimellitate (“CAT”); and hydropropylmethylcellulose acetate succinate. Examples of suitable polyvinyl-based polymers include polyvinyl acetate phthalate (“PVAP”). The second material is preferably a co-polymer of a (meth)acrylic acid and a (meth)acrylic acid C₁₋₄ alkyl ester, for instance, a copolymer of methacrylic acid and methacrylic acid methyl ester. Such polymers include those available under the Trade Marks Eudragit L, Eudragit S and Eudragit FS. The use of Eudragit S as the film-forming polymeric is particularly preferred.

The “core” is usually a single solid body. The core may consist of 5-ALA alone, optionally with SFC, or may be bead of edible material coated with a layer of the active ingredient. The core may for example include a filler or diluent material, e.g. lactose or cellulose material such as microcrystalline cellulose; a binder, e.g. polyvinylpyrrolidone (PVP); a disintegrant, e.g. croscarmellose sodium; and/or a lubricant, e.g. magnesium stearate. The core may be a compressed granulate comprising one or more of these materials.

In such compositions, multi-unit dosage forms comprising particles having a diameter of less than 3 mm are preferred.

Release of the 5-ALA from the composition is delayed until the lower gastrointestinal tract and preferably the ileum and/or the colon. Such compositions have application in a multi-phasic release composition comprising at least two pluralities of particles, e.g. coated pellets, in the same dosage form, e.g. a capsule, in which the particles of one plurality are differentiated from the particles of the or each other plurality by the coating. The coatings may differ from one plurality to the next in terms of coating thickness or composition, e.g. the ratio and/or identity of components. Multi-phasic release formulations would be particularly suitable for suffers of Crohn's disease affecting different regions along the intestine, including the ileum and/or colon.

Medical Applications

The present invention provides a pharmaceutical composition as defined herein for use in therapy. It also provides a method of treating or preventing a disease or condition in a subject, especially a human subject, which comprises administering to the subject a pharmaceutical composition according to the invention in which 5-ALA is medically indicated for treatment or prevention of said disease or condition. In a preferred embodiment of the invention, the compositions are adapted for administration via the oral or rectal route. The invention finds utility in the treatment of diseases of the intestine and particularly in the treatment of diseases of the ileum and/or the colon. It also has application as a portal for entry of 5-ALA into the systemic circulation by absorption from the large intestine, and particularly the ileum and/or the colon, and hence finds utility in the treatment of a wide range of diseases and conditions. It may for example find utility in the treatment or prevention of autoimmune diseases.

The invention finds particular utility in the treatment or prevention, including maintenance of remission or prevention of relapse, of a disease or condition of the ileum and/or the colon, especially the colon, for example inflammatory bowel disease (including ulcerative colitis and Crohn's disease), constipation, diarrhoea, infection, or cancer. The treatment and/or prevention of IBD is of particular importance.

The following Examples illustrate the invention. The Examples refer to the following figures:

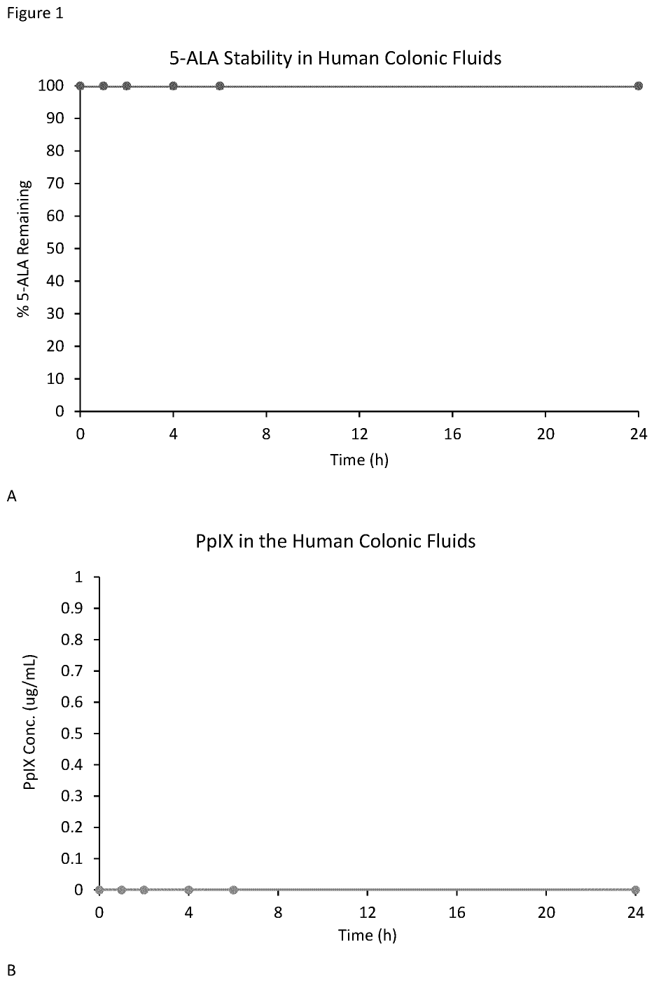

FIG. 1 shows the stability of 5-ALA and formation of PpIX in human colon. A) Stability of 5-ALA in human colon fluid. B) Formation of PpIX after incubation of 5-ALA+SFC in human colon fluid.

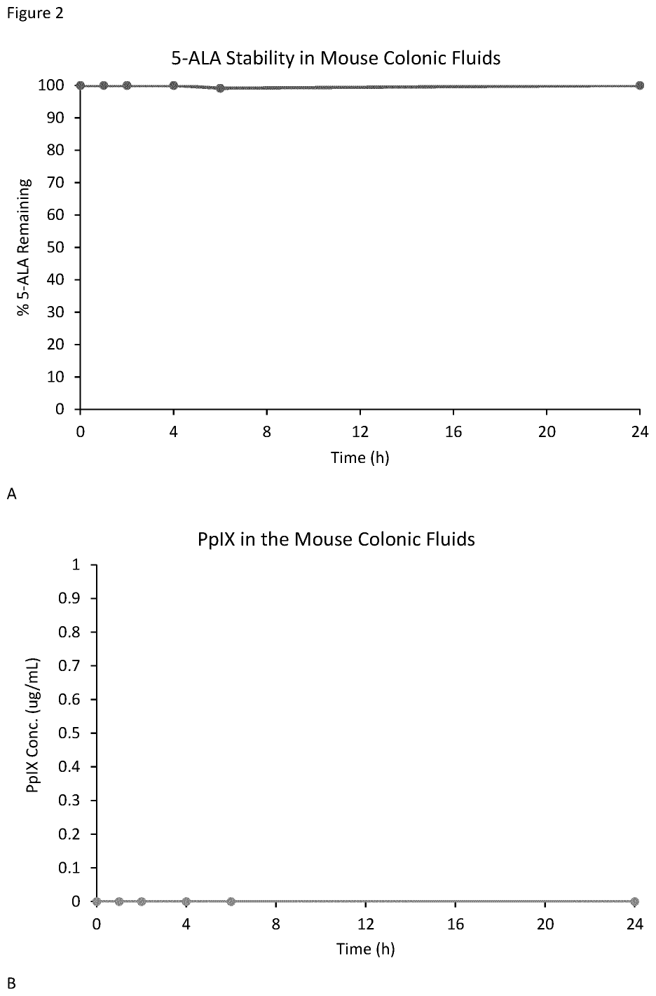

FIG. 2 shows the stability of 5-ALA and formation of PpIX in mouse colon. A) Stability of 5-ALA in mouse colon fluid. B) Formation of PpIX after incubation of 5-ALA+SFC in mouse colon fluid.

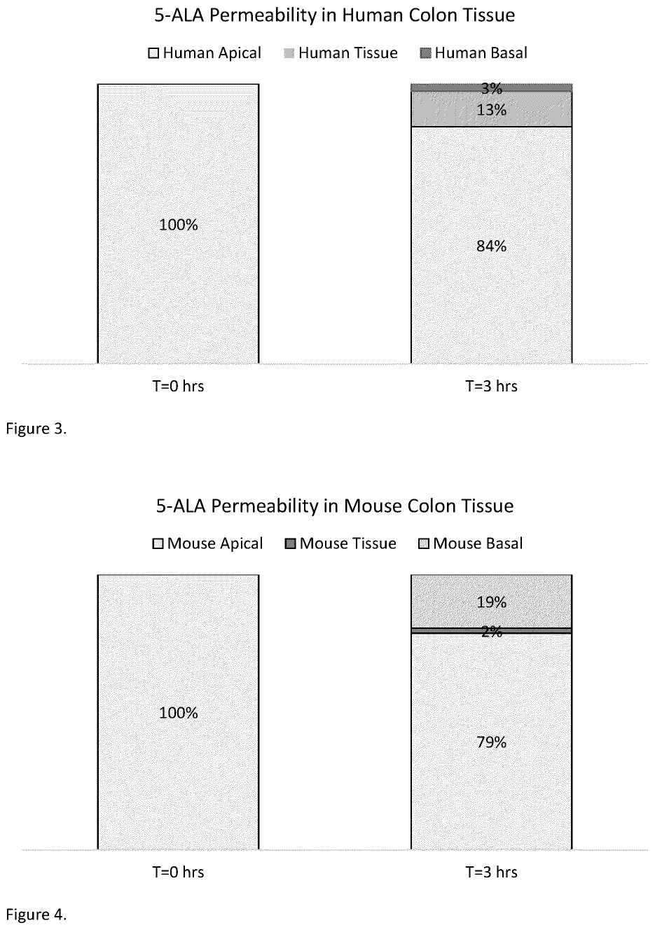

FIG. 3 shows the 5-ALA levels in the apical, tissue and basal compartments after exposure to human colon tissue.

FIG. 4 shows the 5-ALA levels in the apical, tissue and basal compartments after exposure to C57BL6 mouse colon tissue.

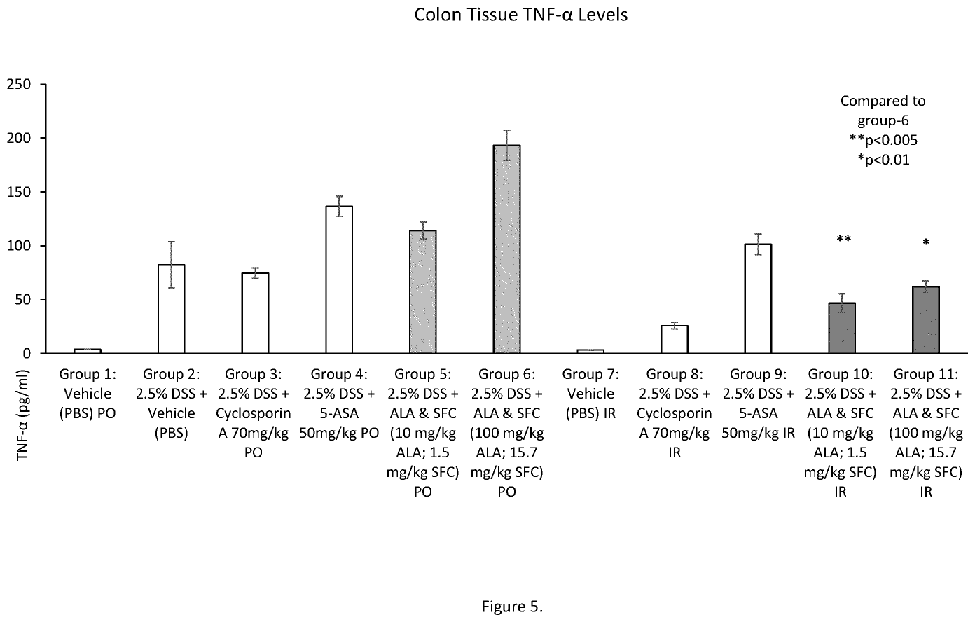

FIG. 5 shows TNF-α levels in mouse colon tissue at 10 days following treatment.

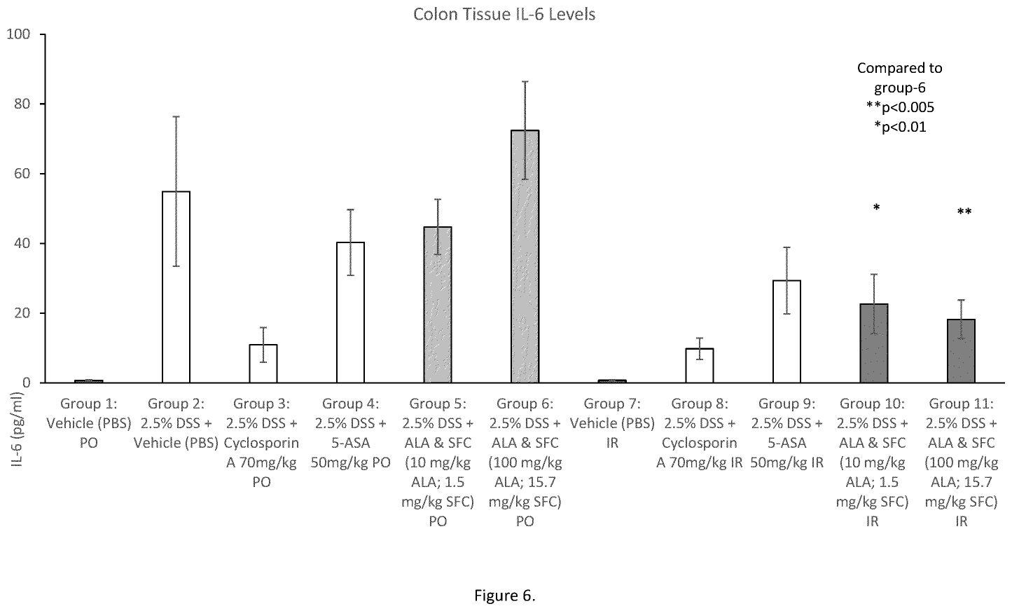

FIG. 6 shows IL-6 levels in levels in mouse colon tissue at 10 days following treatment.

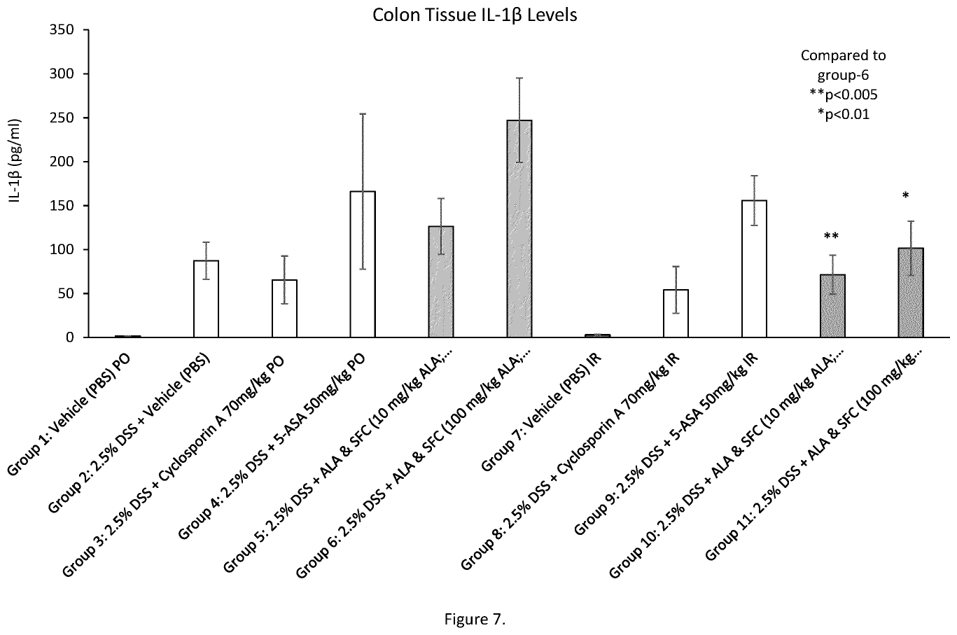

FIG. 7. IL-1β levels in mouse colon tissue at 10 days following treatment.

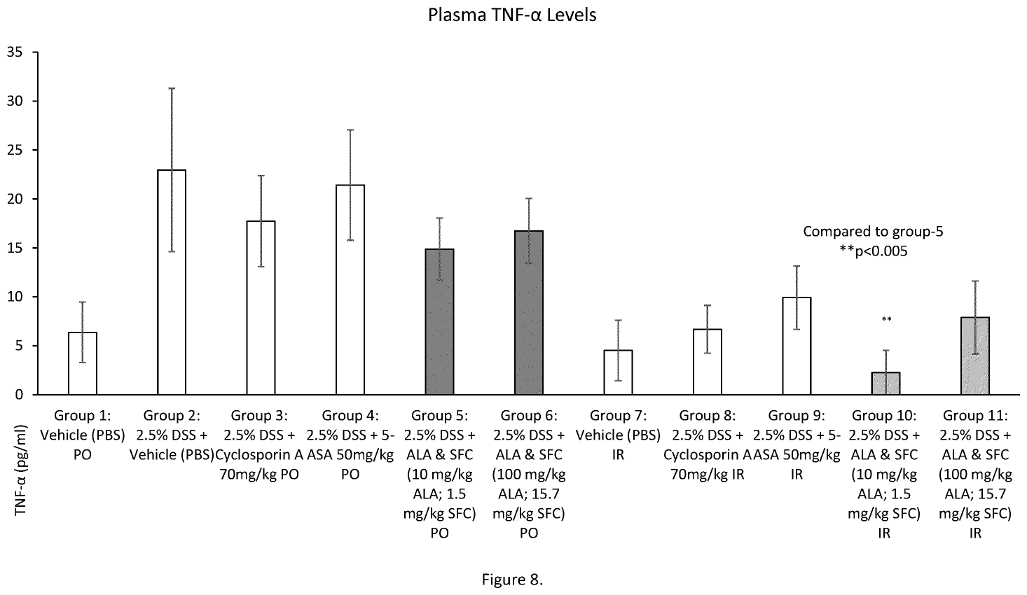

FIG. 8 shows TNF-α levels in mouse colon tissue at 10 days following treatment.

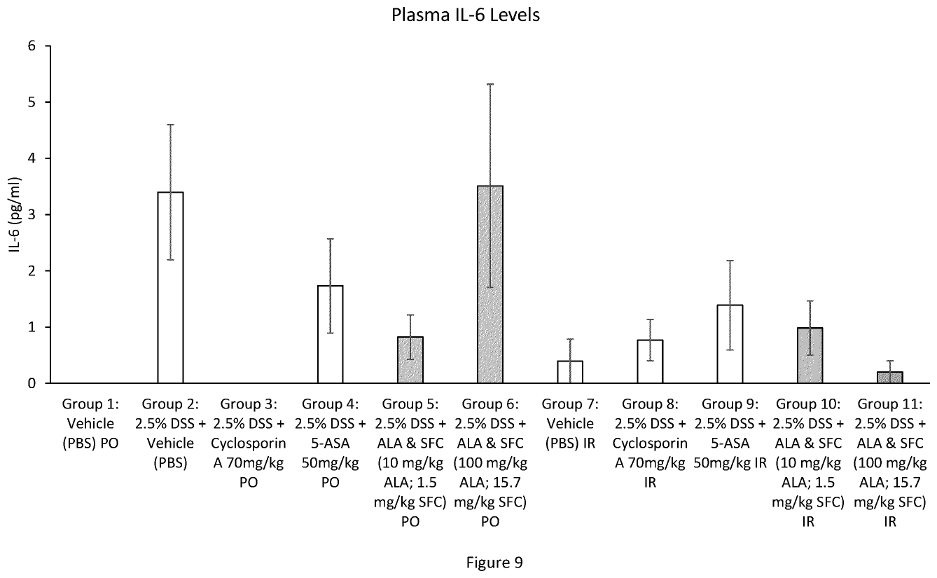

FIG. 9 shows IL-6 levels in mouse colon tissue at 10 days following treatment.

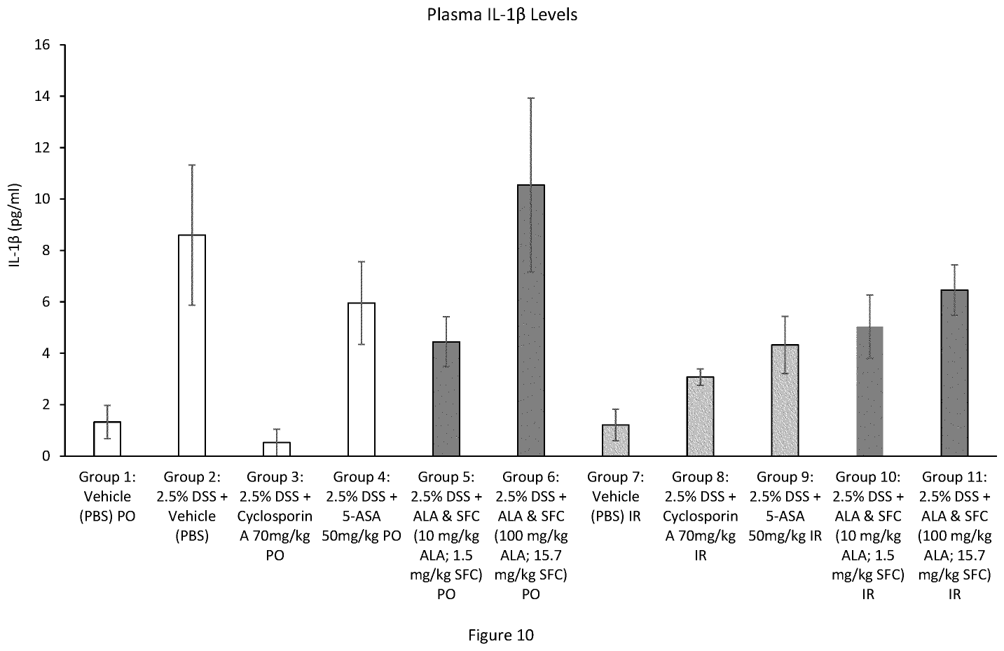

FIG. 10 shows IL-1δ levels in mouse colon tissue at 10 days following treatment.

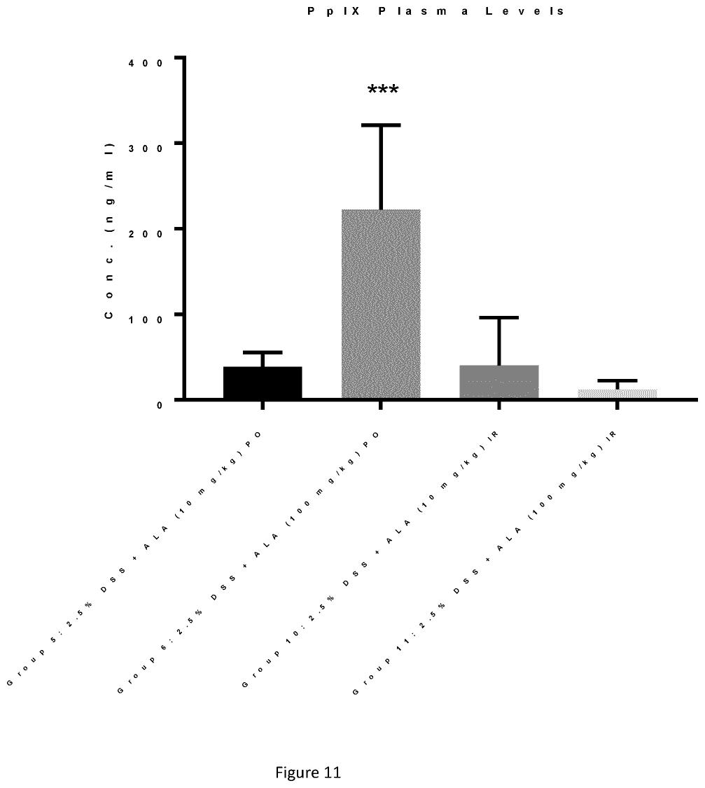

FIG. 11 shows PpIX levels in the plasma at day 10 after 5-ALA dosing orally and intra-rectally at 10 mg/kg and 100 mg/kg doses.

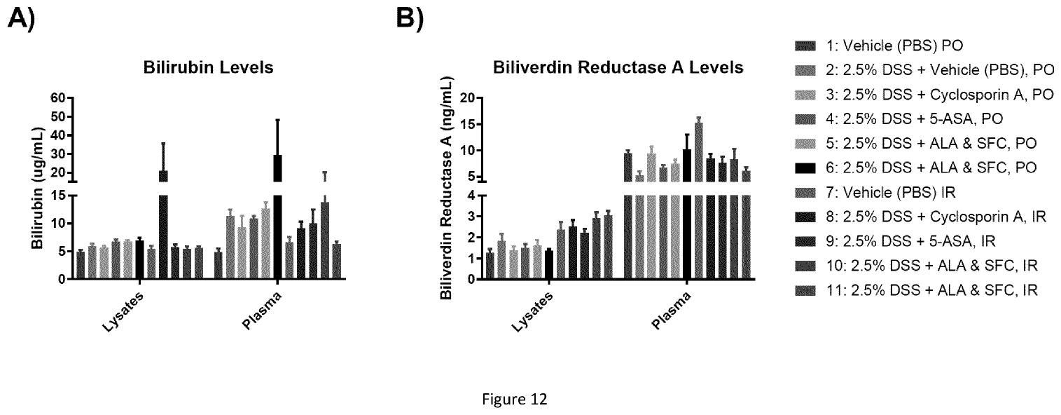

FIG. 12 the levels of metabolites in the tissue and plasma. A) Bilirubin levels in the colon tissue and plasma of mice at day 10 following treatment. B) Biliverdin levels in mouse colon tissue and plasma in all groups at day 10 following treatment.

### MATERIALS AND METHODS

Mouse Colon Model

A mouse colonic model based on a mixed fecal inoculum was used to mimic the luminal environment of the mouse large intestine. An anaerobic workstation (Electrotek 500TG™ workstation, Electrotek, West Yorkshire, UK) maintained at 37° C. and 70% relative air humidity was used to set up the model. Three healthy male C57BL6 mice were sacrificed and the fecal contents were collected. The fecal material was transferred in the anaerobic workstation and diluted with freshly prepared basal medium to obtain 20% w/w slurry by homogenization. The basal media provides nutrients and growth factors to the microbiota allowing viability for upto 24 hours. The homogenized bacterial media was sieved through an open mesh fabric (SefarNitex™, pore size 350 μm) to remove any nonhomogeneous fibrous material.

Human Colon Model

A human colonic model based on a mixed fecal inoculum was used to mimic the luminal environment of the human large intestine. An anaerobic workstation (Electrotek 500TG™ workstation, Electrotek, West Yorkshire, UK) maintained at 37° C. and 70% relative air humidity was used to set up the model. The fecal material was transferred in the anaerobic workstation and diluted with freshly prepared basal medium to obtain 20% w/w slurry by homogenization. The basal media provides nutrients and growth factors to the microbiota allowing viability for up to 24 hours. The homogenized bacterial media was sieved through an open mesh fabric (SefarNitex™, pore size 350 μm) to remove any nonhomogeneous fibrous material. The pH was maintained at approximately 7 to mimic the colonic luminal pH of the human.

5-ALA+SFC Incubation Studies and Processing of Solution for Analysis of 5-ALA and PpIX

5-ALA and SFC stock solution was prepared in PBS at 12 mg/ml and 2 mg/ml respectively. The stock was added to 20% human or mouse faecal slurry to obtain an incubation concentration of 6 mg/ml 5-ALA and 1 mg/ml SFC, and 10% w/w faecal slurry. Samples were withdrawn at appropriate time points and added to 0.5% TFA in a ratio of 1:2. The samples were centrifuged at 9.6 g for 10 mins and the supernatant was prepared for detection of 5-ALA and PpIX by HPLC-FLD.

For detection of 5-ALA, the solution containing 5-ALA was added to 0.1% fluorescamine solution and borate buffer solution in the ratio of 1:1 and 1:3 respectively. The solution was vortexed and incubated for 10 minutes at room temperature before added into the HPLC for analysis.

PpIX solution was prepared by adding the supernatant in an extraction solvent consisting of 100 parts of N,N-dimethylformamide and 1 part of 2-proponol. The mixture was vortexed and transferred for analysis by HPLC-FLD.

HPLC-FLD for 5-ALA and PpIX

Sample analysis for detecting of 5-ALA and PpIX was performed using a high performance liquid chromatography (HPLC) system (Agilent Technologies, 1260 Infinity II Series™) equipped with a pump (model G1311C), autosampler (model G1329B) and a diode-array UV detector (model G1314B).

For 5-ALA analysis, a 150×4.6-mm Jupiter 5 μm 300 Å (Phenomenex, Torrance, Calif.) C18 column was used using 70% water (0.1% TFA) and 30% acetonitrile (0.1% TFA) as the mobile phase for elution, at a flow rate of 1 ml/min. The analysis was operated at room temperature and fluorescence detection wavelength was set at 395/480 nm excitation/emission. The injection volume of each sample was 10 μl.

For PpIX analysis, a 150×4.6-mm Aeris 3.6 μm 100 Å (Phenomenex, Torrance, Calif.) C18 column was used using 70% acetonitrile and 30% 10 mM TBA solution (pH 7.5) as the mobile phase at a flow rate of 1 ml/min. The analysis was operated at room temperature and fluorescence detection wavelength was set at 400/630 nm excitation/emission. The injection volume of each sample was 50 μl.

Ussing Chamber System

A NaviCyte vertical ussing system (Harvard Apparatus, Cambridge, UK) was used to measure transport across epithelial membranes which are polar structures possessing an apical (mucosal) and basolateral (serosal) side. The chambers are made of solid acrylic and supports the tissue membrane in such a way that each side of the membrane is isolated and faces a different chamber representing the luminal (apical) and blood (basal) compartments. The working system consists of a unit to fit a maximum of six vertical chambers, a gas manifold for carbogen purging (95% O₂, 5% CO₂) and a heater block to maintain the temperature of the chambers at 37° C. during the experiments with the use of a circulating water bath. The chambers are two-piece assemblies held together by a high spring-tension retaining ring to ensure leak-free operation during the experiments.

The EVOM™ voltohmmeter (World Precision Instruments, Inc., Hertfordshire, UK) and Ag/AgCl electrodes (Harvard Apparatus, Cambridge, UK) were used to measure the trans-epithelial electrical resistance (TEER) of the tissue samples. TEER monitors the presence of functional tight junctions, which are responsible for the barrier function and which limit Paracellular permeation of water and solutes. TEER value of 200 Ω/cm² was set as the lower limit to confirm the tissue viability and tight junction integrity.

For the tissue penetration studies, the freshly excised colon of human subject or C57BL6 mice was collected and transferred to an ice-cold solution of Krebs-Bicarbonate Ringer solution (KBr) of pH 7.4. The tissue was cut open transversally and was washed with KBr solution to remove the luminal contents and was then mounted in the Ussing chambers. The mucosal surface of the colon tissue was facing the apical chamber, and the endothelial surface of the tissue was facing the basolateral chamber. The exposed tissue area on each side of the chamber was 0.29 cm² and the tissue mounting region was 4×8 mm (FIG. 3.6). The volume of KBr in apical and basolateral chamber was 5 ml and the pH was maintained at 7.4. The tissue was allowed to incubate with KBr for 20 minutes before addition of the drug. 5-ALA and SFC concentrations tested during the penetration experiments was 6 mg/ml and 1 mg/ml respectively. The penetration of 5-ALA and formation of metabolites biliverdin and bilirubin in the tissue was tested for 3 hours and in a minimum of 3 mice. The tissue without drug was incubated in parallel for the same time which acted as the negative control. The chambers were purged with carbogen and kept at 37° C. by water jackets during incubation. The TEER was continuously monitored during the experiment to confirm the viability and integrity of the tissue. Tissues with TEER value below 200 were not used for the experiments.

Tissue Homogenization and 5-ALA, Biliverdin and Bilirubin Extraction for Quantification by HPLC-FLD

Freshly excised colon tissues following completion of the Using chamber permeation study for 3 hrs were weighed and the appropriate amount of extraction buffer was added in the ratio 20 mg: 1 ml. The tissue was homogenized, and the homogenate was incubated for 2 hrs at 4° C. After centrifugation, the supernatant was analyzed for 5-ALA levels as per the protocol described above.

For detection of biliverdin and bilirubin, the samples were added into a dilution buffer (methanol, ammonia solution and water in the ratio of 50:1:49) in a ratio of 1:10. The solution was mixed in the dark and then added to HPLC-FLD for analysis.

HPLC-FLD Analysis of Biliverdin and Bilirubin

A 10×4.6-mm Aeris 3.6 μm 100 Å (Phenomenex, Torrance, Calif.) C18 column was used using 70% acetonitrile and 30% 10 mM TBA solution (pH 7.5) as the mobile phase at a flow rate of 1 ml/min. The analysis was operated at room temperature and fluorescence detection wavelength was set at 400/630 nm excitation/emission. The injection volume of each sample was 50 μl.

In-Vivo Anti-Inflammatory Efficacy of 5-ALA with SFC in DSS Colitis Mouse Model

ALA with SFC, the positive controls 5-aminosalicylic acid (5-ASA) or Cyclosporin A were dosed prophylactically either orally or intra-rectally once daily in C57BL/6 mice (male, 9 weeks old) that received treatment of 2.5% DSS in their drinking water. Animals were kept on 2.5% DSS for 7 days and then switched to normal water for 3 days before being sacrificed.

The different groups in the study were as follows:

| | Treatment | Test Article | Test Article | |
|---|---|---|---|---|
| Group | Description | Dose (mg/kg) | Conc. (mg/ml) | N |
| 1 | Vehicle (PBS) | N/a | N/a | 7 |
| 2 | 2.5% DSS + | N/a | N/a | 7 |
| | Vehicle | | | |
| 3 | 2.5% DSS + | 70 | 7 | 7 |
| | Cyclosporin A | | | |
| 4 | 2.5% DSS + | 50 | 15 | 7 |
| | 5-ASA | | | |
| 5 | 2.5% DSS + | 10 mg/kg ALA + | 3 mg/kg ALA & | 7 |
| | ALA & SFC | 1.5 mg/kg SFC | 0.45 mg/kg SFC | |
| 6 | 2.5% DSS + | 100 mg/kg ALA + | 30 mg/kg ALA + | 7 |
| | ALA & SFC | 15.7 mg/kg SFC | 4.7 mg/kg SFC | |
| 7 | Vehicle (PBS) | N/a | N/a | 7 |
| 8 | 2.5% DSS + | 70 | 7 | 7 |
| | Cyclosporin A | | | |
| 9 | 2.5% DSS + | 50 | 15 | 7 |
| | 5-ASA | | | |
| 10 | 2.5% DSS + | 10 mg/kg ALA + | 3 mg/kg ALA & | 7 |
| | ALA & SFC | 1.5 mg/kg SFC | 0.45 mg/ks SFC | |
| 11 | 2.5% DSS + | 100 mg/kg ALA + | 30 mg/kg ALA + | 7 |
| | ALA & SFC | 15.7 mg/kg SFC | 4.7 mg/kg SFC | |

Groups 1-6 were dosed orally via gavage, while groups 7-10 were dosed intra-rectally directly into the colon.

All groups were evaluated for changes in body weight, stool consistency, stool blood and colon weight/length ration compared to naïve tap-water drinking animals. Colon samples were also collected and subject to histopathology evaluation, cytokine/chemokine and metabolite analysis, and neutrophil myeloperoxidase activity. The study was terminated on Day 10, and necropsy was performed with tissue collection. Blood was collected by terminal cardiac puncture and plasma (>0.2 ml) was isolated by centrifugation, aliquoted into 50 μL aliquots, and stored at −80° C. Plasma was then subjected to chemokine/cytokine analysis of HO-1, TNF-α, IL-1β, IL-6, IL-10, and IL-2 levels as well as analysis of bilirubin and biliverdin levels. In the PK groups, plasma from 2 hours post-dose was analyzed for levels the metabolite PpIX.

### Example 1. Stability of 5-ALA with SFC and Formation of PpIX in Human and Mouse Colon Model

Colon stability was assessed using the human and mouse colon model with the amount of intact 5-ALA remaining and subsequent formation of PpIX at each time point assessed by HPLC-FLD as described in the Methods section. The stability of 5-ALA and formation of PpIX in human and mouse colon are shown in FIGS. 1 and 2 respectively. The results show that 5-ALA is completely stable in both human and mouse colon fluid for up to 24 hrs. However, no conversion of 5-ALA in to PpIX was observed in the presence of SFC in both human and mouse colon.

### Example 2. Human and Mouse Colon Tissue Penetration and Permeability of 5-ALA with SFC and Formation of PpIX, Biliverdin and Bilirubin

Colon tissue penetration and permeability of 5-ALA in the presence of SFC was assessed using healthy human and mouse colon tissue and formation of metabolites PpIX, biliverdin and bilirubin was analysed by HPLC-FLD as described in the Methods section. FIGS. 3 and 4 shows the penetration and permeability of 5-ALA in human and mouse colon tissue respectively. Formation of PpIX, biliverdin and bilirubin are shown in table below:

| | PpIX (μg/ml) | Biliverdin (μg/ml) | Bilirubin (μg/ml) |
|---|---|---|---|
| Human | 0 | 0 | 0 |
| Mouse | 0 | 0 | 0 |

The tissue conc. of 5-ALA was higher in human colon compared to mouse colon, while higher permeability of 5-ALA across the tissue was detected in mouse colon compared to human colon.

### Example 3. In-Vivo Anti-Inflammatory Efficacy of 5-ALA with SFC in DSS Colitis Mouse Model

The ability of 5-ALA to reduce inflammation was investigated in a DSS induced colitis mouse model at 10 and 100 mg/kg dose. The drug was administered orally via gavage and intra-rectally directly into the colon. Expression of key inflammatory cytokines were measured locally in the colon tissue and plasma (FIGS. 5-10). To further understand the differences in the anti-inflammatory mechanisms of oral vs intra-colonic administration of 5-ALA, the formation of active metabolites biliverdin and bilirubin along with PpIX were measured in the tissue and plasma (FIGS. 11 and 12). 5-ALA was able to reduce the expression of inflammatory cytokines TNF-α, IL-1β and IL-6 in the colon tissue and plasma significantly better than after oral administration. The reduction in inflammatory cytokines was also superior to 5-ASA given both orally and intra-colonic. The expression of 5-ALA metabolite PpIX was higher in the plasma when given orally compared to intra-colonic, however, no difference in biliverdin and bilirubin expression was measured in the colon tissue and plasma after oral and intra-colonic dosing of 5-ALA. The data in the present invention suggests that the superior anti-inflammatory effect of intra-colonic administration of 5-ALA compared to oral administration is independent of SFC and anti-inflammatory metabolites formation (biliverdin and bilirubin) and is a direct anti-inflammatory effect of 5-ALA when administered locally in the inflamed colon. Local delivery of 5-ALA offers a unique therapeutic option for the treatment of inflammatory bowel disease at much lower doses and hence frequency of administration than 5-ASA which is the current standard first line therapy.

1. A pharmaceutical composition comprising 5-aminolevulinic acid (5-ALA) or a pharmaceutically acceptable salt thereof, adapted for topical administration to the lower gastrointestinal tract of a human or animal.

2. A pharmaceutical composition as claimed in claim 1, adapted for rectal administration.

3. A pharmaceutical composition as claimed in claim 1, adapted for delayed release oral administration.

4. A pharmaceutical composition as claimed in claim 3, comprising an enteric coating.

5. A pharmaceutical composition as claimed in claim 3, comprising a solid oral dosage form with a core and a coating for the core, the core comprising 5-ALA or a pharmaceutically acceptable salt thereof, and the coating comprising a mixture of a digestible polysaccharide and a film-forming material which has a pH threshold at pH 5.0 or above,

6. A pharmaceutical composition as claimed in claim 5 wherein the digestible polysaccharide is selected from the group consisting of starch; amylose; amylopectin; chitosan; chondroitin sulfate; cyclodextrin; dextran; pullulan; carrageenan; scleroglucan; chitin; curdulan and levan.

7. A pharmaceutical composition as claimed in claim 5, in which the polysaccharide is starch, amylose or amylopectin.

8. A pharmaceutical composition as claimed in either claim 5 or claim 6, in which the film-forming is an acrylate polymer, a cellulose polymer or a polyvinyl-based polymer.

9. A pharmaceutical composition as claimed in claim 7, in which the film-forming is selected from cellulose acetate phthalate; cellulose acetate trimellitate; hydropropylmethylcellulose acetate succinate; and polyvinyl acetate phthalate.

10. A pharmaceutical composition as claimed in claim 8, in which the film-forming is a co-polymer of a (meth)acrylic acid and a (meth)acrylic acid C₁₋₄ alkyl ester.

11. A pharmaceutical composition as claimed in any preceding claim, for use in the prophylaxis or treatment of inflammatory bowel disease, irritable bowel disease, autoimmune disease, constipation, diarrhoea, infection, or cancer.

12. A pharmaceutical composition for use of claim 11, wherein the inflammatory bowel disease is Ulcerative colitis or Crohn's disease.

13. A method of treating inflammatory bowel disease, irritable bowel disease, autoimmune disease, constipation, diarrhoea, infection, or cancer comprising topically administering 5-aminolevulinic acid (5-ALA) or a pharmaceutically acceptable salt thereof, to the lower gastrointestinal tract of a human or animal.

14. A method of claim 13 comprising administering a pharmaceutical composition as defined in any one of claims 1 to 12.

15. A method of claim 13 or claim 14 wherein the inflammatory bowel disease is Ulcerative colitis or Crohn's disease.
